(19) United States
(12) Patent Application Publication
(10) Pub. No.: US 2016/0008295 A1
(43) Pub. Date: Jan. 14, 2016

(54) COMPOSITIONS AND METHODS FOR TREATING ROSACEA

(71) Applicant: TissueTech, Inc., Doral, FL (US)

(72) Inventors: Scheffer TSENG, Pinecrest, FL (US); Sean TIGHE, Tampa, FL (US)

(21) Appl. No.: 14/798,807

(22) Filed: Jul. 14, 2015

Related U.S. Application Data
(60) Provisional application No. 62/024,262, filed on Jul. 14, 2014.

(51) Int. Cl. A61K 31/045; A61K 47/10; A61K 47/38; A61K 47/34; A61K 45/06; A61K 9/70

(52) U.S. Cl. CPC A61K 31/045; A61K 45/06; A61K 9/70; A61K 47/38; A61K 47/34; A61K 47/10

(57) ABSTRACT

The present invention relates to terpine-4-ol containing pharmaceutical compositions, dermatological delivery systems and methods of treatment that are useful for treating rosacea, to include ocular rosacea, papulopustular rosacea, phymatous rosacea, acne rosacea, rosacea associated with Demodex infections, erythematelangiectatic rosacea, steroid induced rosacea, and combinations thereof, acne vulgaris and related conditions in a patient in need thereof.

### CROSS REFERENCE TO RELATED APPLICATION

This application claims the benefit under 35 U.S.C. 119(e) of U.S. Provisional Application No. 62/024,262, filed Jul. 14, 2014 entitled “COMPOSITIONS AND METHODS FOR TREATING ROSACEA,” the disclosure of which is incorporated by reference in its entirety.

### FIELD

The present application describes pharmaceutical compositions containing Terpinen-4-ol (hereinafter “T4O”), dermatological delivery systems containing the pharmaceutical compositions and methods of using the pharmaceutical compositions to treat rosacea.

### BACKGROUND

Rosacea is a chronic dermatological disease that affects the skin, usually the face, and sometimes the eyes. Inflammatory rosacea causes persistent redness and pink bumps referred to as papules, and pustules on the skin. Eye inflammation also may occur, with symptoms often including sensitivity to light, blurred or otherwise impaired vision, redness, dryness, itching, burning, tearing, and the sensation of having grit or sand in the eye. Inflammation of the eye is more apparent in advanced stages of rosacea, where the skin thickens and becomes a deep shade of red. Current treatments include oral antibiotics, e.g., tetracycline or doxycycline. If infections of the eyelids develop, physicians may recommend scrubbing the eyelids with diluted baby shampoo. Steroid eye drops may be prescribed in the case of severe infection.

### SUMMARY

Compositions are described herein containing about 0.01% to about 20% of T4O. In a further aspect are dermatological delivery systems, where dermatological delivery systems include a dispenser; an inert support in contact with a pharmaceutical composition comprising about 0.01% to about 20% w/w T4O and a pharmaceutically acceptable ointment base; and instructions for use comprising the steps of applying the pharmaceutical composition to the affected area, massaging the pharmaceutical composition onto the affected area and repeating the applying and massaging steps until sufficient to reduce the rosacea symptoms. In some embodiments, such compositions are in the form of solutions, suspensions, spray, lotions, gels, pastes, medicated sticks, balms, cleansers (including shampoos and soaps), creams, or ointments. In some embodiments, the compositions are in the form of an ointment.

In one aspect are dermatologic or ophthalmic compositions comprising about 0.6% to about 20% w/w T4O, about 3.0% to about 15% w/w T4O, about 4% to about 10% w/w T4O, or about 5% T4O, and a dermatologically and/or ophthalmically acceptable base. In some embodiments, the dermatologically and/or ophthalmically acceptable base is an ointment base.

In yet another aspect are methods for treating rosacea, in a subject in need, where the method includes applying a dermatologic composition comprising about 0.6% to about 20% w/w T4O and a dermatologically acceptable base to the affected area; massaging the composition onto the affected area; and repeating the applying and massaging steps until sufficient to show an improvement in the disorder. In some embodiments, the dermatologically acceptable base is an ointment base. In some embodiments, the methods also include scrubbing the affected area with a T4O solution or suspension (e.g., T4O shampoo).

Additionally, compositions are described herein containing about 1% to about 20% T4O, about 0.2% to about 5.6% γ-terpinene, about 0.2% to about 3% 1,8 cineole, or about 0.2% to about 2.6% α-terpinene, or any combination thereof. In a further aspect are articles of manufacture, where an article of manufacture includes a dispenser; a pharmaceutical composition comprising about 1% to about 20% T4O, about 0.2% to about 5.6% γ-terpinene, about 0.2% to about 3% 1,8 cineole, or about 0.2% to about 2.6% α-terpinene, or any combination thereof and a pharmaceutically acceptable ointment base; and instructions for use comprising the steps of applying the pharmaceutical composition to the affected area, massaging the pharmaceutical composition onto the affected area and repeating the applying and massaging steps until sufficient to reduce the redness and itching of the rosacea symptoms. In some embodiments, the pharmaceutical composition comprises about 5% w/w T4O. In some embodiments, such compositions are in the form of solutions, suspensions, spray, lotions, gels, pastes, medicated sticks, balms, cleansers (including shampoos and soaps), creams, or ointments. In some embodiments, the compositions are in the form of an ointment.

In one aspect are dermatologic or ophthalmic compositions comprising about 1% to about 20% T4O, about 0.2% to about 5.6% γ-terpinene, about 0.2% to about 3% 1,8 cineole, or about 0.2% to about 2.6% α-terpinene, or any combination thereof, and a dermatologically and/or ophthalmically acceptable base. In some embodiments, the dermatologically and/or ophthalmically acceptable base is an ointment base.

In a further aspect are methods for treating rosacea, in a patient in need thereof, where the methods include applying a dermatologic composition comprising about 1% to about 20% T4O, about 0.2% to about 5.6% γ-terpinene, about 0.2% to about 3% 1,8 cineole, or about 0.2% to about 2.6% α-terpinene, or any combination thereof, and a dermatologically acceptable base to the affected area; massaging the composition onto the affected area; and repeating the applying and massaging steps until sufficient to show an improvement in the disorder. In some embodiments, the methods further include scrubbing the affected area with a T4O solution or suspension (e.g., T4O shampoo).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The appended claims particularly point out features set forth herein. A better understanding of the features and advantages of the present disclosure will be obtained by reference to the following detailed description that sets forth illustrative embodiments, in which the principles described herein are utilized.

Disclosed herein are pharmaceutical compositions containing T4O, dermatological delivery systems containing these pharmaceutical compositions and methods for their use in treating Rosacea (including, ocular rosacea, papulopustular rosacea, phymatous rosacea, acne rosacea, rosacea associated with Demodex infections, erythematelangiectatic rosacea, steroid induced rosacea, and combinations thereof), acne vulgaris and related conditions.

Rosacea is commonly treated with systemic (oral tetracycline antibiotics) and topical (metronidazole antibiotics) drugs. Additionally, ocular rosacea can be treated by performing a daily eyelid margin scrub with diluted shampoo in combination with warm compresses. Further, dermatological vascular laser machines may be used to treat rosacea. They use light to penetrate the epidermis to target the capillaries in the dermis layer of the skin. The light is absorbed by oxy-hemoglobin, which heat up, causing the capillary walls to heat up to 70° C. (158° F.), damaging them, causing them to be absorbed by the body's natural defense mechanism. Unfortunately, these treatments frequently fail to eradicate the disorder and the conditions persist.

Accordingly, described herein is a new method for treating the aforementioned conditions. Without wishing to be bound by theory, it is believed that the compositions described herein alleviate the symptoms of rosacea due to the enhanced skin penetration of the active components. Thus, the compositions of the present application are formulated such that an effective amount of the active agent penetrates the skin on an individual in need of treatment without causing irritation.

The epidermis is the outer layer of the skin, serving as the physical and chemical barrier between the interior body and exterior environment; the dermis is the deeper layer providing the structural support of the skin. The main barrier in skin permeation is the layer of dead cells—the stratum corneum of the epidermis (mainly lipophilic)—and topically applied substances have basically three possibilities to penetrate into the skin: transcellular, intercellular, and follicular. (Trommer 2006, Overcoming the Stratum Corneum Modulation of Skin Penetration). The principal mode of action that enables drugs to diffuse through skin may be described by the lipid-protein partitioning theory. According to this theory, penetrators act by disrupting the highly ordered lipid structure between the corneacytes, interacting with intracellular proteins to disorganize molecular packing or increased partitioning of the drug into the tissue.

The compositions, dermatological delivery systems and methods described herein can be used to treat conditions that include, but are not limited to, rosacea (including, ocular rosacea, papulopustular rosacea, phymatous rosacea, acne rosacea, rosacea associated with Demodex infections, erythematelangiectatic rosacea, steroid induced rosacea, and combinations thereof), acne vulgaris and combinations thereof. Rosacea symptoms typically manifest as redness on the central face across the cheeks, nose, or forehead, but can also less commonly affect the neck, chest, ears, and scalp. In some cases, additional symptoms, such as semi-permanent redness, telangiectasia (dilation of superficial blood vessels on the face), red domed papules (small bumps) and pustules, red gritty eyes, burning and stinging sensations, and in some advanced cases, a red lobulated nose (rhinophyma), may develop.

Compositions

The agents described herein may be administered topically and can be formulated into a variety of topically administrable compositions comprising an active ingredient and a dermatologically acceptable base and/or an ophthalmically acceptable base. Such compositions can be formulated, e.g., as solutions, suspensions, spray, lotions, gels, pastes, medicated sticks, balms, shampoos, soap bars, liquid soaps, creams or ointments. In one embodiment, the composition is the form of an ointment that can be applied in or around the eye of a mammal, including a human.

The active ingredients of such compositions can include T4O and/or any combination of ingredients found in tea tree oil (see herein).

As used herein, “tea tree oil,” i.e., 100% tea tree oil comprises the ranges of components listed in Table 1.

TABLE 1
COMPONENTS OF TEA TREE OIL (TTO)

| | INGREDIENT | ISO 4730 RANGE (%) |
|---|---|---|
| 1 | Terpinen-4-ol | >30 |
| 2 | γ-Terpinene | 10-28 |
| 3 | 1,8 Cineole | 0-15 |
| 4 | α-Terpinene | 5-13 |
| 5 | p-Cymene | 0.5-12 |
| 6 | α-Terpineol | 1.5-8 |
| 7 | δ-Cadinene | Trace-8 |
| 8 | Aromadendrene | Trace-7 |
| 9 | Ledene | 0.5-6.5 |
| 10 | α-Pinene | 1-6 |
| 11 | Terpinolene | 1.5-5 |
| 12 | Limonene | 0.5-4 |
| 13 | Sabinene | Trace-3.5 |
| 14 | Globulol | Trace-3 |
| 15 | Viridiflorol | Trace-1.5 |

As used herein, a “dermatologically acceptable base,” refers to one or more excipients that combine to form a compositon suitable for topical administration.

As used herein, an “ophthalmically acceptable base,” refers to one or more excipients that combine to form a composition suitable for optical administration.

In some embodiments, a composition contains a dermatologically and/or ophthalmically acceptable base and about 0.01% to about 20% (w/w) T4O, e.g., about 3% to about 15%, about 4% to about 10%, about 5%, or any other percent (w/w) of T4O from about 0.01% to about 20%, e.g., 0.20%, 0.22% 0.30%, 0.40%, 0.50%, 0.60%, 0.80%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.7%, 5.2%, 5.7%, 6.2%, 6.7%, 7.2%, 7.7%, 8.2%, 8.7%, 9.2%, 9.5%, 10.0%, 10.5%, 11.2%, 11.5%, 12.0%, 12.4%, 12.9%, 13.3%, 13.5%, 13.7%, 14.0%, 14.5%, 15.0%, 15.5%, 16.0%, 16.6%, 17.0%, 17.4%, 18.0%, 18.5%, 18.8%, 19.0%, 19.2%, 19.4%, 19.5%, 19.6%, 19.7%, 19.8%, 19.9%, or 20% T4O (w/w).

In some embodiments, a composition comprises a dermatologically and/or ophthalmically acceptable base and about 1% to about 20% (w/w) terpinen-4-ol, e.g., about 3% to about 15%, about 4% to about 10%, about 5%, or any other percent (w/w) of terpinen-4-ol falling between about 1% to about 20%, e.g., 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.5%, 5.2%, 5.7%, 6.2%, 6.7%, 7.4%, 7.7%, 8.2%, 8.5%, 9.2%, 9.5%, 10.0%, 10.5%, 11.2%, 11.5%, 12.0%, 12.4%, 12.9%, 13.3%, 13.5%, 13.7%, 14.0%, 14.7%, 15.0%, 15.5%, 16.0%, 16.6%, 17.0%, 17.4%, 18.3%, 18.5%, 18.8%, 19.0%, 19.2%, 19.4%, 19.5%, 19.6%, 19.7%, 19.8%, 19.9%, or 20% terpinen-4-ol.

In some embodiments, a composition comprises a dermatologically and/or ophthalmically acceptable base and about 0.2% to about 9% (w/w) γ-Terpinene, e.g., about 0.4% to about 5.2%, about 0.7% to about 5%, about 0.9% to about 4.4%, about 1.3% to about 4%, about 1.5% to about 3.6%, or any other percent (w/w) of terpinen 4-ol from about 0.2% to about 5.6%, or any other percent (w/w) of γ-Terpinene from about 1% to about 6%, e.g., 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.5%, 5.2%, 5.7% 6.2%, 6.7%, 7.4%, 7.7%, 8.2%, or 8.5% γ-Terpinene.

In some embodiments, a composition comprises a dermatologically and/or ophthalmically acceptable base and about 0.2% to about 10% (w/w) 1,8 cineole, e.g., about 0.4% to about 2.6%, about 0.6% to about 2.4%, 0.8% to about 2.2%, about 0.9% to about 2.0%, or any any other percent (w/w) of 1,8 cineole from about 0.2% to about 3%, e.g., 0.20%, 0.22% 0.30%, 0.40%, 0.50%, 0.60%, 0.80%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.7%, 5.2%, 5.7%, 6.2%, 6.7%, 7.2%, 7.7%, 8.2%, 8.7%, 9.2%, or 9.5% 1,8 cineole.

In some embodiments, a composition comprises a dermatologically and/or ophthalmically acceptable base and about 0.2% to about 4.5% (w/w) α-terpinene, e.g., about 0.3% to about 2.2%, about 0.5% to about 2.0%, about 0.6% to about 1.8%, or any other percent (w/w) of α-Terpinene from about 0.1% to about 4.5%, e.g., 0.20%, 0.22% 0.30%, 0.40%, 0.50%, 0.60%, 0.80%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2% α-terpinene.

In some embodiments, a composition comprises a pharmaceutically acceptable ointment and a combination two or more of about 0.01% to about 20% (w/w) terpinen 4-ol, 0.2% to about 5.6% (w/w) γ-Terpinene; about 0.2% to about 3% (w/w) 1,8 cineole; and about 0.3% to about 2.6% (w/w) α-terpinene.

In some embodiments, a dermatologically and/or ophthalmically acceptable base includes a pharmaceutically acceptable ointment base. Examples of suitable ointment bases include, but are not limited to oleaginous ointment bases such as petrolatum (e.g., liquid petrolatum or white petrolatum), plastibase, hard paraffin, white soft paraffin, yellow soft paraffin, liquid paraffin, emulsifying wax, microcrystalline wax, white bees wax, yellow bees wax, carnauba wax, wool wax (wool fat), mineral oil, olive oil, purified lanolin, anhydrous lanolin, and water soluble ointment bases such as polyethylene glycol (e.g., polyethylene glycol 400 or polyethylene glycol 3350), propylene glycol, polyoxyethylene, polyoxypropylene, or any combinations thereof.

In some embodiments, a dermatologically and/or ophthalmically acceptable base includes one or more polymers as suspending agents. Useful polymers include, but are not limited to, water-soluble polymers such as cellulosic polymers, e.g., hydroxypropyl methylcellulose, and water-insoluble polymers such as cross-linked carboxyl-containing polymers. A dermatologically and/or ophthalmically acceptable base can also include a dermatologically and/or ophthalmically acceptable mucoadhesive polymer, e.g., carboxymethylcellulose, carbomer (acrylic acid polymer), carbopol (copolymers or acrylic acid crosslinked with a polyakenyl polyether), poly(methylmethacrylate), polyacrylamide, polycarbophil, acrylic acid/butyl acrylate copolymer, sodium alginate, or dextran.

In some embodiments, a dermatologically and/or ophthalmically acceptable base includes one or more viscosity enhancing agents. Examples of suitable viscosity enhancing agents include, but are not limited to, methyl cellulose, xanthan gum, gum tragacanth, carboxymethyl cellulose, silica, silicone, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, acacia, corn starch, gelatin, or combinations thereof.

In some embodiments, a dermatologically and/or ophthalmically acceptable base includes one or more dermatologically and/or ophthalmically acceptable pH adjusting agents or buffering agents, including, but not limited to, acids such as acetic, boric, citric, lactic, phosphoric and hydrochloric acids; bases such as sodium hydroxide, sodium phosphate, sodium borate, sodium citrate, sodium acetate, sodium, lactate and tris-hydroxymethylaminomethane; and buffers such as citrate/dextrose, sodium bicarbonate and ammonium chloride. Such acids, bases and buffers are included in an amount required to maintain pH of the composition in a dermatologically and/or ophthalmically acceptable range.

In some embodiments, a dermatologically and/or ophthalmically acceptable base includes one or more dermatologically and/or ophthalmically acceptable salts in an amount required to bring osmolality of the composition into a dermatologically and/or ophthalmically acceptable range. Such salts include, but are not limited to, those having sodium, potassium or ammonium cations and chloride, citrate, ascorbate, borate, phosphate, bicarbonate, sulfate, thiosulfate or bisulfite anions; specific salts include, e.g., sodium chloride, potassium chloride, sodium thiosulfate, sodium bisulfite, and ammonium sulfate.

In some embodiments, a dermatologically and/or ophthalmically acceptable base includes one or more dermatologically and/or ophthalmically acceptable preservatives to inhibit microbial activity. Suitable preservatives include, but are not limited to, mercury-containing substances such as merfen and thiomersal; stabilized chlorine dioxide; and quaternary ammonium compounds such as benzalkonium chloride, cetyltrimethylammonium bromide and cetylpyridinium chloride.

In further embodiments, a dermatologically and/or ophthalmically acceptable base includes one or more dermatologically and/or ophthalmically acceptable surfactants to enhance physical stability, or for other purposes. Suitable nonionic surfactants include isohexadecane, cyclomethicone, copolymers of ethylene glycol and propylene glycol, polyoxyethylerie fatty acid glycerides and vegetable oils, e.g., polyoxyethylene (60) hydrogenated castor oil; and polyoxyethylerie alkylethers and alkylphenyl ethers, e.g., octoxynol 10, octoxynol 40.

In further embodiments, a dermatologically and/or ophthalmically acceptable base includes one or more dermatologically and/or ophthalmically acceptable penetration enhancers to enhance physical stability, or for other purposes. Penetration enhancers are substances which enhance passage of topically-applied compounds into the stratum, corneum of the skin and therefrom into the epidermis and dermis. Examples include, but are not limited to: dimethyl isosorbide, ethoxydiglycol, 1-dodecylazacycloheptan-2-one, propylene glycol, oleyl alcohol, polyoxyethylene ester, sorbitan mono-9-octadecenoate, poly(oxy-1,2-ethanediyl) and derivatives thereof, ethanol, glyceryl monoethyl ether, monoglycerides, isopropylmyristate, lauryl alcohol, lauric acid, lauryl lactate, terpinol, menthol, D-limonene, beta-cyclodextrin, DMSO (dimethyl sulfoxide), polysorbates, fatty acids (e.g., oleic), bile salts, N-methylpyrrolidone, polyglycosylated glycerides, 1-dodecylazacycloheptan-2-one (Azone®), Cyclopentadecalactone (CPE-215®), Alkyl-2-(N,N-disubstituted amino)-alkanoate ester (NexAct®), 2-(n-nonyl)-1,3-dioxolane (DEPA®), and penetration enhancers shown for example in U.S. Pat. Nos. 3,909,816; 4,405,616; 4,801,586; 4,861,764; 4,886,783; 4,983,396; 5,118,845; 5,196,410, 8,486,374 and 8,741,265, each of which is hereby expressly incorporated herein by reference in its entirety.

In further embodiments, a dermatologically and/or ophthalmically acceptable base includes one or more dermatologically and/or ophthalmically acceptable permability enhancers to enhance physical stability, or for other purposes. A variety of classes of compounds may serve as suitable permeability enhancers according to the invention. A first category includes fatty acids and salts and esters thereof, including mono-, di-, and triglycerides. Medium chain length fatty acids, especially C8 and C10 acids, and their salts and esters are particularly useful. Suitable specific examples include sodium caprylate, sodium caprate, CAPMUL® glycerides (available from Abitec of Columbus, Ohio), LABRASOL® glycerides (PEG-8 caprylic/capric glycerides, available from Gattefosse SAS of Saint Priest, Cedex, France), GELUCIRE® 44/14 (PEG-32 glyceryl laurate EP, available from Gattefosse), other glycerides & fatty acid esters, CREMOPHOR® (BASF, Ludwigshafen, Germany), D-α-tocopheryl polyethylene glycol 1000 succinate, vegetable oils, polyoxylglycerides, and medium chain mono- and diacylglycerides.

One example of this class, CAPMUL® MCM L8 (glycerol monocaprylate) (available from Abitec of Columbus, Ohio), is composed of mono- and diglycerides of medium chain fatty acids (mainly caprylic, with some capric) and 7% maximum free glycerol. It contains at least 44% alpha monoglycerides (as caprylate).

Other examples of this class of enhancers include GATTEFOSSE compositions 61A through 61H which are proprietary to Gattefosse SAS, but generally are composed of mixtures containing one or more of medium chain mono-, di-, or triglycerides, polysorbate derivatives, polyoxyl castor oil derivatives, polyethylene glycol derivatives including polyethylene glycol glycerides, polyoxyl ethers, vegetable oils, glycerin, and similar GRAS (generally regarded as safe) lipidic components in varying amounts. These components are part of individual commercial products such as CAPRYOL™ 90, CAPRYOL™ PGMC, LAUROGLYCOL™ 90, GELUCIRE® 44/14, Plurol Oleique CC497, LABRASOL®, LABRAFIL® M1944CS (apricot kernel oil PEG-6 esters), Transcutol HP, Peceol, and Maisine 35-1, all of which are available from Gattefosse SAS.

While not falling directly within this class, glycerol itself has been found to impart excellent permeability enhancement, particularly for neuraminidase inhibitors. This result was not anticipated as glycerol is not considered a permeability enhancer.

A second category of enhancers includes surfactants having a steroidal structure, such as bile acid salts. Examples of suitable compounds include sodium cholate, sodium deoxycholate, glycocholate, glycoursodeoxycholate, taurocholate, taurodeoxycholate, and steroid detergents/bile salts. Other surfactants may also be suitable permeability enhancers, including cationic, anionic, and nonionic surfactants. Examples include polysorbate 80, hexadecyldimethylbenzylammonium chloride, N-hexadecylpyridinium bromide, dodecyltrimethylammonium bromide, hexadecyltrimethylammonium bromide, tetradecyl-β-D-maltoside, octylglucoside, glycyrrhetinic acid, 3-(N,N-dimethylpalmitylammonio)propane-sulfonate, and sodium lauryl sulfate.

Cyclodextrins may also be used as suitable enhancers. Examples include β-cyclodextrin, hydroxypropyl-β-cyclodextrin, γ-cyclodextrin, and hydroxypropyl-γ-cyclodextrin.

A variety of other compounds may also be used as enhancers. Examples include sodium salicylate, ethylenediamine tetraacetic acid (EDTA), citric acid, chitosan & chitosan derivatives, N-trimethyl chitosan chloride, monocarboxymethyl-chitosan, palmitoyl carnitine chloride, acyl carnitines, ethylene glycol tetraacetic acid (EGTA), 3-alkylamido-2-alkoxypropyl-phosphocholine derivatives, alkanoylcholines, N-acetylated amino acids (based on α- and non-α-amino acids), mucoadhesive polymers, phospholipids, piperine, 1-methylpiperazine, α-amino acids, and mineral oil.

Thus a wide variety of enhancer compounds may be selected from the group consisting of fatty acids, fatty acid esters, fatty acid salts, glycerol, surfactants, cyclodextrins, sodium salicylate, ethylenediamine tetraacetic acid, citric acid, chitosan, chitosan derivatives, N-trimethyl chitosan chloride, monocarboxymethyl-chitosan, palmitoyl carnitine chloride, acyl carnitines, ethylene glycol tetraacetic acid, 3-alkylamido-2-alkoxypropyl-phosphocholine derivatives, alkanoylcholines, N-acetylated amino acids, mucoadhesive polymers, phospholipids, piperine, 1-methylpiperazine, α-amino acids, and mineral oil.

The permeability enhancer and the polar agent may be mixed in any proportion so long as there is provided a therapeutically effective amount of the polar agent and a permeability-enhancing amount of the enhancer compound. Enhancement in dermal bioavailability of topically administered polar agents can depend on the nature and concentration of the enhancer compound with which the agent is formulated. It is thus contemplated that the required therapeutic amount may be contained in a single dosage form or divided between one or more dosages intended for application at the same time or in sequence.

The permeability enhancers act relatively independently of the concentration of polar agent. Differing permeability enhancers can reach either optimal or maximum enhancement over a wide concentration range depending on their particular inherent enhancement potential. Often, enhancers have a non-linear dose response relationship between concentration of enhancer present and amount of increased polar agent absorption. The amount of enhancer to be utilized in an oral dosage form with a polar agent is initially based upon the enhancement properties observed in Caco-2 cell assays at varying fixed enhancer concentrations. Based upon those results, an effective in vivo amount of enhancer compound for a human formulation can be estimated, demonstrated and optimized without undue experimentation using methods well known to those skilled in the formulation art, to achieve a desired pharmacokinetic in vivo profile.

In formulating the composition of this invention, it will be apparent to those skilled in the formulation art that more effective enhancer compounds would require less polar agent than less effective permeability enhancers to achieve a target pharmacokinetic profile. Given those considerations and variations, the amount of enhancer may be at least about 0.1 wt % of the combined weight of enhancer and polar agent, more preferably at least about 50 wt %, and more preferably at least 70 wt % of the combined weight of enhancer and polar agent. The amount is preferably at most 95 wt %, more preferably at most 80 wt %, and more preferably at most 75 wt % of the combined weight of the enhancer and polar agent. Thus, as shown in the examples, a typical dosage form may contain a wide range of concentrations of enhancer compounds depending on the compound itself and its efficacy in enhancing the permeability of polar agents following oral administration. Concentrations as low as 0.001% by weight up to 20% have been demonstrated to be effective in enhancement of the permeability of polar agents.

In yet other embodiments, a dermatologically and/or ophthalmically acceptable base includes one or more antioxidants to enhance chemical stability where required. Suitable antioxidants include, by way of example only, butylated hydroxytoluene (BHT), sodium ascorbate, ascorbic acid, sodium metabisulfite, and tocopherol. In certain embodiments, antioxidants enhance chemical stability where required.

In addition to those enumerated above, any other surfactant, moisturizer, gelling agent, preservative, colorant or pigment, antioxidant, radical scavenger, emulsifier, humectant, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein.

Well-known carriers used to formulate other topical therapeutic compositions for administration to humans will be useful in these compositions. Examples of such components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

Methods for determining the presence and amounts of specific chemical components and byproducts thereof (e.g., degradation products) in any of the compositions described herein include, for example, an assay method can be based on the industry standard produced by the Australian Standard Method as 2782-1997, “Oil of Melaleuca, terpinen-4-ol type (Tree Tea Oil)” and following GLP set using Gas Chromatography (GC-FID) and Gas-chromatography mass spectrometry. See, e.g., Brophy et al. (1989), J Agric Food Chem, 37:1330-1335; and Mondello et al. (2006), BMC Infect Dis, 6:158.

The composition may be used immediately or stored for later use in any type of container known to one of skill in the art such as, for example, pouch, jar, bottle, tube, ampule and pre-filled syringe. Finally, the composition may be sterilized by any method known to one of skill in the art such as, for example, γ radiation.

Toxicity and therapeutic efficacy of such compositions can be determined by standard pharmaceutical procedures in cell cultures or experimental animals, for example, in accordance with the ISO 10993-1 standard for toxicology testing and in accordance with GLP (Good Laboratory Practice) regulations.

For example, cell culture assays can be used to assess the biocompatibility of a material through the use of isolated cells in vitro. These techniques are useful in evaluating the toxicity or irritancy potential of materials and chemicals and they provide an excellent way to screen material prior to in vivo tests. Specifically, the MEM elution assay can be performed on a series of dilutions of the compounds described herein. Each compond dilution is added to a monolayer of L-929 cells and then incubated. Afterwards, cells are examined microscopically for malformation, degeneration and lysis, and the test compound is scored for its cytopathic effect.

In another example, an ocular irritation test is designed to determine the ocular irritation and toxicity of solutions for up to 72 hours in rabbits' eyes. Generally, 3 rabbits with clinically normal eyes are used in a study. Rabbits' eyes are examined daily and scored using the Draize system. Before treatment and at 1, 24, 48 and 72 hours, the eyes of each rabbit are also examined with an ophthalmoscope and scored for ocular irritation using the McDonald-Shadduck method (slit-lamp and fluorescein stain).

Methods of Treatment

The compositions described herein can be used for the manufacture of a medicament for treating any of the foregoing conditions e.g., Rosacea, to include ocular rosacea, papulopustular rosacea, phymatous rosacea, acne rosacea, rosacea associated with Demodex infections, erythematelangiectatic rosacea, steroid induced rosacea, and combinations thereof, acne vulgaris and combinations thereof.

In some embodiments, where the condition to be treated is Rosacea, to include ocular rosacea, papulopustular rosacea, phymatous rosacea, acne rosacea, rosacea associated with Demodex infections, erythematelangiectatic rosacea, steroid induced rosacea, and combinations thereof, acne vulgaris and combinations thereof, or a related condition, the compositions described herein (e.g., a dermatogically or ophthalmically acceptable ointment) are administered (e.g., self administered) topically by gentle application to a subject's skin, or, if treating an ocular condition, the eyelid margin, skin, and eyelash roots, followed by massaging of the eyelid margin and skin from one end to the other. In some embodiments, excess composition is left on the eyelid area until the next treatment. In other embodiments, excess composition is wiped or washed away after massaging. In some embodiments, the eyelid margin, skin, and eyelash root areas are scrubbed with a T4O solution or suspension prior to application of one of the ophthalmically acceptable compositions described herein. In other embodiments, the T4O solution or suspension is used to scrub after one of the ophthalmically acceptable compositions described herein has been applied and massaged onto the eyelid margin, skin, eyelash root areas. The T4O solution or suspension used for scrubbing can have any concentration of T4O from about 2% to 100% T4O, e.g., about 2%, 3%, 4%, 4.5%, 5%, 6%, 7%, 9.5%, 12%, 14.5% 17%, 19.5%, 22%, 24.5%, 27%, 29.5%, 32%, 34.5%, 37%, 39.5%, 42%, 44.5%, 47%, 49.5%, 52%, 54.5%, 57%, 60%, 62%, 64.5%, 67%, 69.5%, 72%, 74.5%, 77%, 79.5%, 82%, 84.5%, 87%, 89.5%, 92%, 94.5%, 97%, or 99.5% T4O. In some embodiments, the T4O solution is a T4O shampoo, which is commercially available, e.g., Kato Sales, Inc. (Altamonte Springs, Fla., USA).

A number of endpoints can be used to evaluate the therapeutic efficacy of the methods described herein. For example, a reduction in one or more of skin blotches, swelling, inflammation, vascularity, skin redness, number of papules and/or pustules, itching, dry eye, light sensitivity or eye redness are indicative of a successful treatment. Thus, in some embodiments, applying and massaging of an ointment to the affected area is repeated until one or more of the just-described endpoints are attained.

In an exemplary embodiment, an ointment formulation is generated by mixing T4O with Vaseline to a final concentration (w/w) of 5% T4O Application and massage of 5% T4O ointment is performed twice a day (e.g., once before noon and once before bedtime) for five minutes each, for a total treatment period of about four weeks.

The compositions containing the compound(s) described herein can be administered for prophylactic and/or therapeutic treatments. In therapeutic applications, the compositions are administered to a patient already suffering from Rosacea, to include ocular rosacea, papulopustular rosacea, phymatous rosacea, acne rosacea, rosacea associated with Demodex infections, erythematelangiectatic rosacea, steroid induced rosacea, and combinations thereof, acne vulgaris and combinations thereof, or a related condition, in an amount and duration of application time sufficient to cure or at least partially arrest the symptoms of the disease or condition. Amounts effective for this use will depend on the severity and course of the disease or condition, previous therapy, the patient's health status, weight, and response to the drugs, and/or the judgment of a treating physician.

In prophylactic applications, compositions containing the compounds described herein are administered to a patient susceptible to or otherwise at risk of Rosacea, to include ocular rosacea, papulopustular rosacea, phymatous rosacea, acne rosacea, rosacea associated with Demodex infections, erythematelangiectatic rosacea, steroid induced rosacea, and combinations thereof, acne vulgaris and combinations thereof, or a related condition.

Once improvement of the patient's conditions has occurred based on an evaluation of one or more of the symptoms described herein, a maintenance dose of the composition is administered if necessary. Subsequently, the dosage or the frequency of administration, or both, can be reduced, as a function of the symptoms, to a level at which the improved condition is retained. Patients can, however, require intermittent treatment on a long-term basis upon any recurrence of symptoms.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation.

Combination Treatments

Compositions described herein can also be used in combination with other therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes.

In certain instances, it may be appropriate to administer at least one composition described herein in combination with another therapeutic agent. By way of example only, if one of the side effects experienced by a patient upon receiving one of the compounds described herein is skin irritation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used.

Therapeutically-effective dosages can vary when the drugs are used in treatment combinations, and methods such as (by way of example only) metronomic dosing, i.e., providing more frequent, lower doses in order to minimize toxic side effects, can be used to determine such doses. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more other agents, the compound provided herein may be administered either simultaneously with the agent(s), or sequentially.

In any case, the multiple therapeutic agents (one of which is a composition described herein) may be administered in any order or even simultaneously. If simultaneously, the multiple therapeutic agents may be provided in a single, unified form, or in multiple forms (by way of example only, either as a single ointment or as an ointment and a pill). One of the therapeutic agents may be given in multiple doses, or both may be given as multiple doses. If not simultaneous, the timing between the multiple doses may vary from more than fifteen minutes to less than four weeks. In addition, the combination methods, compositions and formulations are not to be limited to the use of only two agents; the use of multiple therapeutic combinations are also envisioned.

The dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents.

Exemplary additional therapeutic agents may be selected from the group consisting of: an anti-acne agents, an anti-microbial agents, insecticides, antiparasitics agents, anti-inflammatory agents, immunoregulators, antibiotics, bacteriocidal drugs, bacteriostatic drugs, cleansing agents, absorbents, astringents, emollients, moisturizers, keratolytics, retinoids, and anti-fungal agents, salts thereof, and mixtures thereof.

When the additional therapeutic agent is an anti-acne agent it may be selected from the group consisting of: salicylic acid, benzoyl peroxide, adapalene, azelaic acid, clarithromycin, clindamycin, doxycycline, minocycline, topicycline, tetracycline, erythromycin, a macrolide antibiotic, a retinoid, isotretinoin, retinol, T4O, tazarotene, Vitamin A, ciprofloxacin, metronidazole, and tretinoin.

When the additional therapeutic agent is an anti-fungal agent it may be selected from the group consisting of: imidazoles, hydroxy pyridones, triazoles, allyl amines, undecylenic acids, tolnaftate, haloprogin, pyridinethiones, cloquinol, amphotericin B, butoconazole nitrate, ciclopirox olamine, clindamycin, clioquinol, clotrimazole, econazole, econazole nitrate, fluconazole, flucytosine, griseofulvin, itraconazole, ketoconazole, miconazole, micronazole, naftifine, nystatin, omadine disulfide, sulconazole, terbinafine, terconazole, tioconazole, tolnaftate, triacetin, undecylenic acid, zinc pyrithione, efinacoloazole, and mixtures thereof.

When the additional therapeutic agent is an anti-microbial agent it may be selected from the group consisting of: amikacin, bacitracin, colistin, gentamicin, kanamycin, metronidazole, mupirocin, neomycin, netilmicin, polymyxin B, streptomycin, tobramycin, phenols and cresols such as 2,4-dichloro-sym-metaxylenol, parachlorometaxylenol, and parachlorometacresol, bisphenols such as hexachlorophene, dichlorophene, bithionol, triclosan, and fentichlor, salicylanilides such as 4′,5-dibromsalicylanilide, 3′,4′,5-trichlorosalicylanilide, 3′,4′,5-tribromosalicylanilide, and 3,5,dibromo-3′-trifluoromethyl-salicylanilide, carbanilides such as trichlorocarbanilde and 3-trifluoromethyl-4-4′-dichlorocarbanilide, quaternary ammonium compounds such as alkyl-dimethyl benzyl ammonium chloride, alkyl-trimethyl ammonium chloride, alkyl trimethyl ammonium bromide, cetyl-trimethyl ammonium bromide, B-phenoxyethyl-dimethyl-dodecyl ammonium bromide, p-tert-octylphenoxyethoxyethyl-dimethyl-benzyl ammonium chloride, tetradecyl-pyridinium bromide, cetyl pyridinium bromide, cetyl pyridinium chloride, di-(n-octyl)-dimethyl ammonium bromide, alkyl-isoquinolinium bromide, 1-(3-chloroallyl)-3-5-7-triaza-1-azoniaadamantane chloride, and chlorhexidine (1,6,di(N-p-chlorophenylguanidino)hexane), 2-bromo-2-nitropropan-1,3-diol, imidazonidyl urea, ethanol, isopropyl alcohol, natural oils, aqueous and organic extracts of natural or synthetic substances, tea tree oil, and mixtures thereof.

When the additional therapeutic agent is an anti-inflammatory agent it may be selected from the group consisting of: glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics; antibiotics; tarcolimus, retinoids and mixtures thereof.

Articles of Manufacture

For use in the applications described herein, kits and articles of manufacture are also described herein. The terms “kit” and “article of manufacture” are used as synonyms. Such kits can include a carrier, package, or container that is compartmentalized to receive one or more containers such as vials, tubes, and the like, each of the container(s) including one of the separate elements to be used in a method described herein. Preferably, containers (e.g., vials) containing a composition described herein are light-proof have a tight seal. For example, the container(s) can include one of the dermatologically or opthalmically acceptable compositions described herein, i.e., a dermatologically or opthalmically acceptable composition comprising 0.01% to 20% w/w T4O. In an exemplary embodiment, the containers contain a pharmaceutical composition comprising about 5% w/w T4O, as disclosed herein. Suitable containers include, for example, bottles, vials, syringes, and test tubes. The containers can be formed from a variety of materials such as glass or plastic. Preferably, the container protects against certain wavelengths of light, prolonged high temperature, leaching and/or the ingress of air. Preferably the container is a sealed, light-proof container.

The articles of manufacture provided herein contain packaging materials. Packaging materials for use in packaging pharmaceutical products include, by way of example only U.S. Pat. Nos. 5,323,907, 5,052,558 and 5,033,252. Examples of pharmaceutical packaging materials include, but are not limited to, blister packs, bottles, tubes, pumps, bags, vials, light-tight sealed containers, syringes, bottles, and any packaging material suitable for a selected formulation and intended mode of administration and treatment. A wide array of topical formulations of the compounds and compositions provided herein are contemplated as are a variety of treatments for any of the diseases, disorders or conditions described herein.

Such kits optionally comprise a compound with an identifying description or label or instructions relating to its use in the methods described herein. For example, the kit may include instructions for use comprising the steps of applying the pharmaceutical composition to the affected area, massaging the pharmaceutical composition onto the affected area and repeating the applying and massaging steps until reduction of the symptoms occurs.

A kit may include one or more additional containers, each with one or more of various materials desirable from a commercial and user standpoint for use of the compositions for treating any of the diseases, disorders or conditions described herein. Non-limiting examples of such materials include, but not limited to, buffers, diluents, carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling format approved by the U.S. Food and Drug Administration for prescription drugs, over the counter drugs and cosmetics or the approved product insert. Compositions containing a compound provided herein formulated in a compatible pharmaceutical carrier can also be prepared, placed in an appropriate container, and labeled for treatment of an indicated condition.

### EXAMPLES

The following proposed formulation examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Reference thereto evidences the availability and public dissemination of such information.

### Example 1

| | % W/W |
|---|---|
| T4O | 10.0 |
| PEG 400, USP | 54.0 |
| PEG 3350, USP | 36.0 |

### Example 2

| | % W/W |
|---|---|
| T4O | 5.00 |
| Purified Water | 85.0 |
| Hydroxyethyl Cellulose | 3.00 |
| Propylene Glycol | 5.00 |
| Benzyl Alcohol | 2.00 |

### Example 3

| | % W/W |
|---|---|
| T4O | 1.00 |
| Carbopol 934P | 2.00 |
| Pluracare L-62 | 0.500 |
| Glycerin | 4.00 |
| Syloid 244FP | 0.25 |
| Dow Fluid 200 (100Cs) | 0.10 |
| Sodium Hydroxide | q.s. pH about 5 |
| Purified Water | ca. 92.00 |

### Example 4

| | % W/W |
|---|---|
| T4O | 20.0 |
| Propylene Glycol | 40.0 |
| Transcutol | 5.00 |
| Glycerin | 25.0 |
| Oleic Acid | 5.00 |
| Lactic Acid | 5.00 |

### Example 5

| | % W/W |
|---|---|
| T4O | 2.50 |
| Mineral Oil | 3.00 |
| Tefose 63 | 20.0 |
| Labrifil M 1944 CS | 3.00 |
| Benzyl Alcohol | 2.00 |
| Purified Water | q.s. ca. 69.5 |

### Example 6

| | % W/W |
|---|---|
| T4O | 10.00 |
| Cetostearyl Alcohol | 8.00 |
| Propylene Glycol | 6.00 |
| Cyclomethicone | 0.10 |
| Isopropyl Myristate | 13.0 |
| Transcutol | 5.00 |
| Cetomacrogol 1000 | 3.00 |
| Polysorbate 80 | 2.00 |
| Carbomer 934P | 0.50 |
| Methylparaben | 0.15 |
| Propylparaben | 0.02 |
| Sodium Hydroxide | q.s. pH about 5 |
| Purified Water | q.s. ca. 52.23 |

### Example 7

| | % W/W |
|---|---|
| T4O | 5.00 |
| Diisopropyl Adipate | 20.0 |
| Propylene Glycol | 10.0 |
| Polysorbate 20 | 5.00 |
| Polyolprepolymer-15 | 2.00 |
| Sorbitan Monolaurate | 1.00 |
| Carbomer 940P | 0.30 |
| Povidone K-30, USP | 0.25 |
| Trolamine | q.s. pH about 5 |
| Purified Water | q.s. ca 56.00 |

### Example 8

| | % W/W |
|---|---|
| T4O | 10.0 |
| Dimethyl Isosorbide | 20.0 |
| Promulgen G | 7.00 |
| Simethicone | 0.100 |
| Transcutol | 3.00 |
| Purified Water | q.s. ca 59.9 |

### Example 9

| | % W/W |
|---|---|
| T4O | 10.0 |
| Carbopol934P | 2.0 |
| Pluracare L-62 | 0.5 |
| Glycerin | 4.0 |
| Syloid 244FP | 0.25 |
| Dow Fluid 200 | 0.1 |
| Sodium Hydroxide | q.s. pH about 5 |
| Purified Water | q.s. ca 83.0 |

### Example 10

| | % W/W |
|---|---|
| T4O | 10.0 |
| Carbopol934P | 2.0 |
| Transcutol | 5.0 |
| Pluracare L-62 | 0.5 |
| Glycerin | 4.0 |
| Syloid 244FP | 0.25 |
| Dow Fluid 200 | 0.1 |
| Sodium Hydroxide | q.s. pH about 5 |
| Purified Water | q.s. ca 78.0 |

### Example 11

| | % W/W |
|---|---|
| T4O | 10.0 |
| Carbopol934P | 2.0 |
| DMI | 5.0 |
| Pluracare L-62 | 0.5 |
| Glycerin | 4.0 |
| Syloid 244FP | 0.25 |
| Dow Fluid 200 | 0.1 |
| Sodium Hydroxide | q.s. pH about 5 |
| Purified Water | q.s. ca 78.0 |

### Example 12

| | % W/W |
|---|---|
| T4O | 10.0 |
| Carbopol934P | 2.0 |
| Transcutol | 5.0 |
| DMI | 5.0 |
| Pluracare L-62 | 0.5 |
| Glycerin | 4.0 |
| Syloid 244FP | 0.25 |
| Dow Fluid 200 | 0.1 |
| Sodium Hydroxide | q.s. pH about 5 |
| Purified Water | q.s. ca 73.0 |

It is understood that the proposed formulation examples and embodiments described herein are for illustrative purposes only and that various modifications or changes in light thereof will be suggested to persons skilled in the art and are to be included within the spirit and purview of this application and scope of the appended claims.

What is claimed is:

1. A pharmaceutical composition useful for the treatment of rosacea, comprising:
a. from about 0.01% to about 20% w/w terpinen-4-ol; and
b. at least one pharmaceutically acceptable, excipient, diluent or carrier.

2. The composition of claim 1, wherein at least one of the pharmaceutically acceptable excipients, diluents or carriers is selected from the group consisting of a humectant, an inorganic salt, fragrance, dye, colorant, stabilizer, surfactant, moisturizer, emulsifier, pH modifier, solvent, chelate, preservative, water softening agent, thickener and mixtures thereof.

3. The composition of claim 1, wherein at least one of the pharmaceutically acceptable excipients, diluents or carriers is selected from the group consisting an odor masking agent, a color-masking agent or combinations thereof.

4. The composition of claim 1, wherein the composition is a dermatologically or opthamologically acceptable formulation selected from the group consisting of: cream, ointment, lotion, aerosol, solution, suspension, cleanser, soap, foam, mascara, pomade, applicator pencil, gel and other dosage form or device suitable to apply the material.

5. The composition of claim 1, where the pH range of the formulation is between about 3.0 and about 9.0.

6. The composition of claim 1, where the pH range of the formulation is between about 5.0 and about 8.0.

7. The composition of claim 1, wherein the formulation is administered to a patient in need from an applicator.

8. The composition of claim 1, wherein the rosacea is selected from the group consisting of: ocular rosacea, papulopustular rosacea, phymatous rosacea, acne rosacea, rosacea associated with Demodex infections, erythematelangiectatic rosacea, steroid induced rosacea and acne vulgaris.

9. The composition of claim 1, further comprising a least one penetration enhancer selected from the group consisting of: propylene glycol, methanol, water, ethanol, decanol, azones, esters, fatty acids, pyrrolidine, bisabolol, pentylene glycol, dimethyl isosorbide, terpenes, ethoxydiglycol, 1-dodecylazacycloheptan-2-one, oleyl alcohol, polyoxyethylene ester, sorbitan mono-9-octadecenoate, poly(oxy-1,2-ethanediyl), glyceryl monoethyl ether, glycerin, monoglycerides, isopropylmyristate, lauryl alcohol, lauric acid, lauryl lactate, terpinol, menthol, D-limonene, beta-cyclodextrin, dimethyl sulfoxide, polysorbates, bile salts, N-methylpyrrolidone, polyglycosylated glycerides, cyclopentadecalactone, alkyl-2-(N,N-disubstituted amino)-alkanoate ester, 2-(n-nonyl)-1,3-dioxolane, and any combination thereof.

10. The composition of claim 1, further comprising at least one additional therapeutic agent.

11. The composition of claim 10, wherein the at least one additional therapeutic agent is selected from the group consisting of: an anti-acne agent, an anti-microbial agent, insecticides, antiparasitics agents, anti-inflammatory agents, immunoregulators, antibiotics, bacteriocidal drugs, bacteriostatic drugs, cleansing agents, absorbents, astringents, emollients, moisturizers, keratolytics, retinoids, and anti-fungal agents, salts thereof, and mixtures thereof.

12. The composition of claim 11, wherein the anti-acne agent is selected from the group consisting of: salicylic acid, benzoyl peroxide, adapalene, sulfur, azelaic acid, clarithromycin, clindamycin, doxycycline, minocycline, topicycline, tetracycline, erythromycin, a macrolide antibiotic, a retinoid, isotretinoin, retinol, T4O, tazarotene, Vitamin A, ciprofloxacin, metronidazole, and tretinoin.

13. The composition of claim 11, wherein the anti-fungal agent is selected from the group consisting of: imidazoles, hydroxy pyridones, triazoles, allyl amines, undecylenic acids, tolnaftate, haloprogin, pyridinethiones, cloquinol, amphotericin B, butoconazole nitrate, ciclopirox olamine, clindamycin, clioquinol, clotrimazole, econazole, econazole nitrate, fluconazole, flucytosine, griseofulvin, itraconazole, ketoconazole, miconazole, micronazole, naftifine, nystatin, omadine disulfide, sulconazole, terbinafine, terconazole, tioconazole, tolnaftate, triacetin, undecylenic acid, zinc pyrithione, efinacoloazole, and mixtures thereof.

14. The composition of claim 11, wherein the anti-microbial agent is selected from the group consisting of: amikacin, bacitracin, colistin, gentamicin, kanamycin, metronidazole, mupirocin, neomycin, netilmicin, polymyxin B, streptomycin, tobramycin, phenols and cresols such as 2,4-dichloro-sym-metaxylenol, parachlorometaxylenol, and parachlorometacresol, bisphenols such as hexachlorophene, dichlorophene, bithionol, triclosan, and fentichlor, salicylanilides such as 4′,5-dibromsalicylanilide, 3′,4′,5-trichlorosalicylanilide, 3′,4′,5-tribromosalicylanilide, and 3,5,dibromo-3′-trifluoromethyl-salicylanilide, carbanilides such as trichlorocarbanilde and 3-trifluoromethyl-4-4′-dichlorocarbanilide, quaternary ammonium compounds such as alkyl-dimethyl benzyl ammonium chloride, alkyl-trimethyl ammonium chloride, alkyl trimethyl ammonium bromide, cetyl-trimethyl ammonium bromide, B-phenoxyethyl-dimethyl-dodecyl ammonium bromide, p-tert-octylphenoxyethoxyethyl-dimethyl-benzyl ammonium chloride, tetradecyl-pyridinium bromide, cetyl pyridinium bromide, cetyl pyridinium chloride, di-(n-octyl)-dimethyl ammonium bromide, alkyl-isoquinolinium bromide, 1-(3-chloroallyl)-3-5-7-triaza-1-azoniaadamantane chloride, and chlorhexidine (1,6,di(N-p-chlorophenylguanidino)hexane), 2-bromo-2-nitropropan-1,3-diol, imidazonidyl urea, ethanol, isopropyl alcohol, natural oils, aqueous and organic extracts of natural or synthetic substances, tea tree oil, and mixtures thereof.

15. A dermatological delivery system useful for treating rosacea comprising:
a. a topically effective amount of terpinen-4-ol, and
b. an inert support in contact with the terpinen-4-ol

16. The dermatological delivery system of claim 15 wherein the topically effective amount of terpinen-4-ol is
a. a solution comprising:
(1) from about 0.01% to about 20% w/w of terpinen-4-ol; and
(2) at least one pharmaceutically acceptable diluent or carrier.

17. The dermatological delivery system of claim 14, further comprising at least one applicator.

18. The dermatological delivery system of claim 17, wherein the one or more applicators are impregnated with the solution.

19. A dermatological delivery system according to claim 14 wherein said support is a woven fiber matrix.

20. A dermatological delivery system according to claim 19 wherein said support is a non-woven fiber matrix.

21. A dermatological delivery system according to claim 19 wherein said support is a polymeric sponge.

22. A dermatological delivery system according to claim 19 wherein said support is selected from the group consisting of cotton, rayon, polyester, polypropylene, wood pulp, mohair, nylon fleece and neoprene foam, and a combination thereof.

23. The delivery system according to claim 19 wherein said support is rayon and polyester.

24. The delivery system according to claim 23 wherein the support comprises from 20%-80% rayon and from 20%-80% polyester.

25. The delivery system according to claim 24 wherein the support system is 50% polyester and 50% rayon.

26. A dermatological delivery system according to claim 15 wherein said pharmaceutically acceptable diluent or carrier comprises solvents from the group consisting of polar substances, non-polar substances, and mixtures thereof.

27. A dermatological delivery system according to claim 26 wherein said major solvent component is water.

28. The delivery system according to claim 27 wherein the water is present in an amount between 0%-and about 100%.

29. A dermatological delivery system according to claim 26 wherein said major solvent component is a mixture of water and ethanol.

30. A dermatological delivery system according to claim 26 wherein said major solvent component is selected from the group of water, ethanol or mixtures thereof, an emollient, and a penetration enhancer.

31. The delivery system of claim 15 wherein the volume of said terpinen-4-ol composition delivered is from about 0.1 to about 10 ml.

32. The delivery system of claim 31 wherein the volume of said terpinen-4-ol composition delivered is about 5 ml.

33. The delivery system of claim 15 wherein the inert support is from about 0.5 in.² to about 144 in.² in area.

34. The delivery system of claim 33 wherein the inert support is from about 1 in.² to about 4 in.² in area.

35. The delivery system of claim 15 wherein the inert support is from about 1 mil to about 500 mils thick.

36. The delivery system of claim 35 wherein the inert support is from about 5 mils to about 250 mils thick.

37. The delivery system of claim 36 wherein the inert support is from about 10 mils to about 100 mils thick.

38. A dermatological delivery system comprising a topically acceptable, inert support selected from the group consisting of cotton, rayon, polyester, polypropylene, wood pulp, mohair, nylon fleece, and neoprene foam, or combination thereof, impregnated with an about 0.01% to about 20% terpinen-4-ol composition; said composition having a major solvent component selected from the group of water, ethanol or a mixtures thereof, an emollient, and a penetration enhancer, said support being operable to permit application of said composition to the skin.

39. A dermatological delivery system comprising a topically acceptable, inert support selected from the group consisting of woven or non-woven fiber matrix or a polymeric sponge, or combination thereof, impregnated with an about 0.01% to about 20% terpinen-4-ol composition; said composition having a major solvent component selected from the group of water, ethanol or a mixtures thereof, an emollient, and a penetration enhancer, said support being operable to permit application of said composition to the skin.

40. A dermatological delivery system of claim 39 where the delivery system is effective in reducing the number of demodex organisms on or in the affected tissue

41. A dermatological delivery system of claim 39 where the delivery system is effective in reducing the number of bacteria on or in the affected tissue.

42. A dermatological delivery system of claim 39 where the delivery system is effective in reducing the number of fungi on or in the affected tissue.

43. The delivery system of claim 39 wherein the inert support is single use.

44. The delivery system of claim 39 wherein the inert support is part of a multiple dosing device having a storage means for multiple doses of terpinen-4-ol.

45. The delivery system of claim 39 wherein the multiple dosing device contains from 1-250 ml of a terpinen-4-ol composition.

46. The delivery system of claim 39 wherein the multiple dosing device is a dab-o-matic.

47. The delivery system of claim 39 wherein the storage means comprises plastic, glass or metal.

48. The delivery system of claim 39 wherein the storage means comprises one or more of the following: polyester, polypropylene, polyethylene, glass, steel or aluminum.

49. The delivery system of claim 39 wherein the multiple dosing device is pressurized.

50. A dermatological delivery system as in claim 39 in which the delivery system is packaged in a light and/or oxygen blocking barrier.

51. A dermatological delivery system as in claim 50 in which the blocking barrier is selected from at least one of the following: Polyester/Polyethylene/Foil/Barex; Cellophane/Polyester/Foil/Co-extruded Polyethylene; Cellophane/Poly-ethylene/Foil/Polyethylene; Cellophane/Polyethylene/Foil/Surlyn; Polyester/Polyethylene/Foil/Scl air; Cellophane/Polyethylene/Foil/Foil/co-polymer Paper/Polyethylene/Foil/PET; (polyethyleneterephallate)/Polyethylene Paper/Polyethylene/Foil/Co-extrud-ed Polyethylene; Polyester/Polyethylene/Foil/Ethylene Acrylic Acetate/Polyethylene; Polyester/Polyethylene/Foil/Ethylene Methyl Acrylate Polyethylene; PET/Polyethylene/Foil/Barex.

52. The device of claim 39, further comprising one or more applicators selected from the group consisting of: towels and brushes.

53. The device of claim 39, wherein the solution further comprises one or more pharmaceutically-acceptable excipients.

54. A method of treating the skin, hair or a combination thereof of an individual in need, comprising contacting the skin, hair or combination thereof of the individual with the device of claim 39.

55. The method of claim 54, wherein contacting the skin, hair or combination thereof of the individual with the treatment device comprises cleansing the skin prior to application of the terpinen-4-ol composition.

56. The method of claim 54, comprising contacting the skin, hair or combination thereof of the individual wherein the treatment period to show a visual improvement is from 1 to 26 weeks.

57. The method of claim 54, comprising contacting the skin, hair or combination thereof of the individual wherein the posology required to show a visual improvement is from a single to multiple treatments per day.

58. The method of claim 54, wherein said topical composition is formulated for once-per day administration.

59. The method of claim 58, wherein said once-per-day administration occurs in the A.M.

60. The method of claim 58, wherein said once-per-day administration occurs in the P.M.

61. The method of claim 54, wherein said topical composition is formulated for twice-per-day administration.

62. The method of claim 61, wherein said twice-per-day administration occurs once in the A.M. and once in the P.M.

63. The method of claim 54, wherein said topical composition is formulated for more than twice-per-day administration.

64. The method of claim 54, wherein said treatment regimen is directed by a health care. professional.

65. The method of claim 54, wherein the device further comprises one or more applicators.

66. The method of claim 54, wherein the one or more applicators are soaked in the solution.

67. The method of claim 54, wherein the one or more applicators are cotton applicators.

68. The method of claim 54, wherein the one or more applicators are selected from the group consisting of: towels and brushes.

69. The method of claim 54, further comprising treating rosacea in the individual.

70. The method of claim 54, wherein the skin comprises the rosacea affected areas.

71. The method of claim 54, wherein the device further comprises one or more pharmaceutically-acceptable excipients.

72. The method of claim 54, wherein the one or more pharmaceutically-acceptable excipients are selected from the group consisting of: water, saline, corn oil, olive oil, glycerol, petroleum jelly, dextrose, ethanol and a combination thereof.

73. The method of claim 72, wherein the one or more pharmaceutically-acceptable excipients are selected from the group consisting of: water, glycerol and a combination thereof.

74. The method of claim 54, wherein the solution further comprises at least one penetration enhancer selected from the group consisting of: propylene glycol, methanol, water, ethanol, decanol, azones, esters, fatty acids, pyrrolidine, bisabolol, pentylene glycol, dimethyl isosorbide, terpenes, ethoxydiglycol, 1-dodecylazacycloheptan-2-one, oleyl alcohol, polyoxyethylene ester, sorbitan mono-9-octadecenoate, poly(oxy-1,2-ethanediyl), glyceryl monoethyl ether, glycerin, monoglycerides, isopropylmyristate, lauryl alcohol, lauric acid, lauryl lactate, terpinol, menthol, D-limonene, beta-cyclodextrin, dimethyl sulfoxide, polysorbates, bile salts, N-methylpyrrolidone, polyglycosylated glycerides, cyclopentadecalactone, alkyl-2-(N,N-disubstituted amino)-alkanoate ester, 2-(n-nonyl)-1,3-dioxolane, and any combination thereof.

75. A method of treating rosacea in an individual in need thereof, comprising administering to the individual a therapeutically-effective amount of a pharmaceutical composition comprising:
a. about 0.01% up to, but not including, about 20% w/w of terpinen-4-ol as an active agent; and
b. at least one pharmaceutically acceptable excipient.

76. A method of treating the papules and pustules of rosacea in an individual in need thereof, comprising administering to the individual a therapeutically-effective amount of a pharmaceutical composition comprising:
a. about 0.2% up to, but not including, about 20% w/w of terpinen-4-ol as an active agent; and
b. at least one pharmaceutically acceptable excipient.

77. A method of treating the inflammation associated with rosacea in an individual in need thereof, comprising administering to the individual a therapeutically-effective amount of a pharmaceutical composition comprising:
a. about 0.2% up to, but not including, about 20% w/w of terpinen-4-ol as an active agent; and
b. at least one pharmaceutically acceptable excipient.

78. A method of treating the redness and erythema associated with rosacea in an individual in need thereof, comprising administering to the individual a therapeutically-effective amount of a pharmaceutical composition comprising:
a. about 0.2% up to, but not including, about 20% w/w of terpinen-4-ol as an active agent; and
b. at least one pharmaceutically acceptable excipient.

79. A method of treating the edema, telangiectasias, burning, stinging, dryness or a combination thereof associated with rosacea in an individual in need thereof, comprising administering to the individual a therapeutically-effective amount of a pharmaceutical composition comprising:
a. about 0.2% up to, but not including, about 20% w/w of terpinen-4-ol as an active agent; and
b. at least one pharmaceutically acceptable excipient.

80. A method for enhancing the penetration of terpinen-4-ol into the skin of a patient in need thereof, comprising: administering to a patient in need a pharmaceutical composition, comprising:
a. about 0.2% up to, but not including, about 20% w/w of terpinen-4-ol as an active agent;
b. at least one pharmaceutically acceptable excipient; and
c. at least one penetration enhancer.

81. A method of treating the steroid induced inflammation associated with rosacea in an individual in need thereof, comprising administering to the individual a therapeutically-effective amount of a pharmaceutical composition comprising:
a. about 0.2% up to, but not including, about 20% w/w of terpinen-4-ol as an active agent; and
b. at least one pharmaceutically acceptable excipient.
